(19) United States
(12) Patent Application Publication
(10) Pub. No.: US 2018/0000864 A1
(43) Pub. Date: Jan. 4, 2018

(54) HOMEOPATHIC METHOD FOR TREATING COLIC, GAS AND UPSET STOMACH IN INFANTS

(71) Applicants: Jacqueline COURTIOL, Long Beach, CA (US); Timothy LAWRENCE, Long Beach, CA (US)

(72) Inventors: Jacqueline COURTIOL, Long Beach, CA (US); Timothy LAWRENCE, Long Beach, CA (US)

(21) Appl. No.: 15/545,010

(22) PCT Filed: Dec. 10, 2015
(86) PCT No.: PCT/US15/64904

§ 371 Date: Jul. 20, 2017

Related U.S. Application Data
(60) Provisional application No. 62/105,506, filed on Jan. 20, 2015.

(51) Int. Cl. A61K 33/44; A61K 47/10; A61K 36/9068; A61K 36/886; A61K 36/534; A61K 36/53; A61K 36/28; A61K 36/235; A61K 36/185; A61K 47/12; A61K 9/10

(52) U.S. Cl. CPC A61K 33/44; A61K 47/12; A61K 47/10; A61K 36/9068; A61K 36/886; A61K 9/10; A61K 36/53; A61K 36/28; A61K 36/235; A61K 36/185; A61K 36/534

(57) ABSTRACT

The present invention generally relates to homeopathic compositions, and, more particularly, (i) homeopathic compositions comprising a homeopathically potentised form of Passiflora incarnata (Passionflower), and a homeopathically potentised form of Nux Moschata (Nutmeg); or (ii) a homeopathically potentised Carbo Vegetabilis; and a homeopathically potentised Passiflora incarnata; or (iii) a homeopathically potentised Carbo Vegetabilis; and a homeopathically potentised Nux Moschata; and methods of treatment using these homeopathic compositions.

### CROSS-REFERENCE TO RELATED APPLICATIONS

This application claims the benefit of U.S. Provisional Application Ser. No. 62/105,506, entitled “Homeopathic Method for Treating Colic, Gas and Upset Stomach in Infants,” filed on Jan. 20, 2015, the entire content of which is incorporated herein by reference.

### BACKGROUND OF THE INVENTION

Infant colic is associated with inconsolable crying for long periods beginning in the second week after birth, peaking around 6 weeks and subsiding around 4 months of age. Not wishing to be bound by theory, in the 1950's, it was believed by the pediatrician Morris Wessel that infantile colic comprised episodes of crying for more than three hours a day for more than three days a week for three weeks in an otherwise healthy child between the ages of two weeks and four months (Morris A. Wessel et al. Paroxysmal Fussing in Infancy, Sometimes Called “Colic”, Nov. 1, 1954, PEDIATRICS, Vol. 14, No. 5, 421-435). Even though infant colic affects approximately 25% of the pediatric population, colic among infants cannot be attributed to a single cause. Some experts believe that gastrointestinal discomfort is likely to blame.

Nevertheless, colic remains pervasive in pediatric population and the need for treatment continues unabated.

### BRIEF SUMMARY OF THE INVENTION

The present invention generally relates to homeopathic compositions, and, more particularly, to (i) homeopathic compositions comprising a homeopathically potentised form of Passiflora incarnata (Passionflower), and a homeopathically potentised form of Nux Moschata (Nutmeg); or (ii) a homeopathically potentised Carbo Vegetabilis (activated charcoal); or (iii) a homeopathically potentised Carbo Vegetabilis and a homeopathically potentised Passiflora incarnata; or (iv) a homeopathically potentised Carbo Vegetabilis and a homeopathically potentised Nux Moschata; and (v) methods of treatment using any one of these homeopathic compositions. In some embodiments, the homeopathic composition is formulated as a liquid suspension.

In one aspect, the present invention provides a composition comprising a homeopathically potentised form of Passiflora incarnata; and a homeopathically potentised form of

Nux Moschata. In at least one embodiment, the composition comprising homeopathically potentised form of Passiflora incarnata and Nux Moschata comprises at least one homeopathic dilution. In some embodiments, the at least one homeopathic dilution comprises one or more centesimal homeopathic dilutions. In some other embodiments of the composition comprising homeopathically potentised form of Passiflora incarnata and Nux Moschata, the at least one homeopathic dilution comprises one or more decimal homeopathic dilutions.

In one aspect, the present invention provides a composition comprising a composition comprising a homeopathically potentised Carbo Vegetabilis; and a homeopathically potentised Passiflora incarnata. In at least one embodiment, the composition comprising homeopathically potentised form of Carbo Vegetabilis and Passiflora incarnata comprises at least one homeopathic dilution. In some embodiments, the at least one homeopathic dilution comprises one or more centesimal homeopathic dilutions. In some other embodiments of the composition comprising homeopathically potentised form of Carbo Vegetabilis and Passiflora incarnata, the at least one homeopathic dilution comprises one or more decimal homeopathic dilutions.

In another aspect, the present invention provides a composition comprising a homeopathically potentised Carbo Vegetabilis; and a homeopathically potentised Nux Moschata. In at least one embodiment, the composition comprising homeopathically potentised form of Carbo Vegetabilis and Nux Moschata comprises at least one homeopathic dilution. In some embodiments, the at least one homeopathic dilution comprises one or more centesimal homeopathic dilutions. In some other embodiments of the composition comprising homeopathically potentised form of Carbo Vegetabilis and Nux Moschata, the at least one homeopathic dilution comprises one or more decimal homeopathic dilutions.

In another aspect, the present invention provides a homeopathic composition comprising Carbo Vegetabilis having a mean particle size of less than 120 microns and an iodine number in the range of from about 900 mg/g to about 2,300 mg/g inclusive. In some embodiments of the homeopathic composition the carbo vegetablis has a mean particle size of less than 60 microns. In some embodiments of the homeopathic composition at least 90 percent of the particle distribution lies below D90. In some embodiments of the homeopathic composition the carbo vegetablis has a mean particle size of less than 60 microns.

In another aspect, the present invention provides a method of treatment, comprising administering to a subject in need of such treatment any of the compositions according to the present invention, including any one of the compositions according to the exemplary embodiments disclosed herein. In at least one embodiment of the method of treatment, the treatment comprises treating or alleviating colic symptoms in an infant. In at least one embodiment of the method of treatment, the treatment comprises preventing or reducing symptoms associated with infant colic, gastrointentinal gas, including intestinal gas and bloating, upset stomach, heartburn or indigestion in the infant. In at least one embodiment of the method of treatment, the treatment comprises preventing or reducing symptoms associated with infant colic, intestinal gas, bloating, upset stomach, heartburn or indigestion in a pediatric population. In at least one embodiment of the method of treatment, the homeopathic composition is administered in an amount effective for treating, preventing, or reducing colic or symptoms associated with colic, intestinal gas, bloating, upset stomach, heartburn or indigestion in a subject. In at least one embodiment of the method of treatment, the homeopathic composition is administered in an amount effective for preventing or reducing symptoms associated with infant colic, intestinal gas, bloating, upset stomach, heartburn or indigestion in the infant. In at least one embodiment of the method of treatment, the homeopathic composition is administered in an amount effective for preventing or reducing symptoms associated with infant colic, intestinal gas, bloating, upset stomach, heartburn or indigestion in a pediatric population.

### DETAILED DESCRIPTION OF THE INVENTION

The present subject matter will now be described more fully hereinafter with reference to Examples, which illustrate representative embodiments. The present subject matter can, however, be embodied in different forms and should not be construed as limited to the embodiments set forth herein. Rather, these embodiments are provided to describe and enable one of skill in the art. Unless otherwise defined, all technical and scientific terms used herein have the same meaning as commonly understood by one of ordinary skill in the art to which the subject matter pertains. All publications, patent applications, patents, and other references mentioned herein are incorporated by reference in their entireties.

As used herein the terms such as “homeopathic”, and “homeopathic ingredient” are defined in accordance with The Federal Food, Drug and Cosmetic Act (21 U.S.C. 351), which is incorporated herein by reference, and includes those ingredients, agents or drugs used in the practice of homeopathy and listed in the Homeopathic Pharmacopeia of the United States (HPUS), the entirety of which is also incorporated herein by reference.

### I. COMPOSITIONS

In one aspect, the present invention provides a homeopathic composition that exhibits one or more of the aforementioned desirable attributes. In at least one embodiment, an exemplary composition according to the present invention comprises a homeopathically potentised form of Passiflora incarnata and a homeopathically potentised form of Nux Moschata. In at least one embodiment, the composition comprising homeopathically potentised form of Passiflora incarnata and Nux Moschata comprises at least one homeopathic dilution. In some embodiments, the at least one homeopathic dilution comprises one or more centesimal homeopathic dilutions. In some other embodiments of the composition comprising homeopathically potentised form of Passiflora incarnata and Nux Moschata, the at least one homeopathic dilution comprises one or more decimal homeopathic dilutions.

In some preferred embodiments of the composition comprising homeopathically potentised form of Passiflora incarnata and Nux Moschata, the composition includes at least one homeopathic dilution comprising one or more decimal homeopathic dilutions. In some preferred embodiments of the composition comprising homeopathically potentised form of Passiflora incarnata and Nux Moschata, the composition includes diluted homeopathic Nux Moschata having a potency in the range of 3× to 6×, wherein a dilution of n× dilutes one part of the homeopathic Nux Moschata (Nutmeg) with nine parts of the diluent and is succussed, and the combination of dilution and succussion are repeated n times. In some preferred embodiments of the composition comprising homeopathically potentised form of Passiflora incarnata and Nux Moschata, the composition includes diluted homeopathic Nux Moschata having a potency in the range of 2× to 6×, wherein a dilution of n× dilutes one part of the homeopathic Nux Moschata (Nutmeg) with nine parts of the diluent and is succussed, and the combination of dilution and succussion are repeated n times. Whenever it appears herein, a potency range such as “2× to 6×” refers to each value in the given range up to and including 6×-e.g., “Nux Moschata having a potency in the range of 2× to 6× means that Nux Moschata may have any one of the potencies 2×, 3×, 4×, 5×, and 6×. In some preferred embodiments of the composition comprising homeopathically potentised form of Passiflora incarnata and Nux Moschata, the composition includes diluted homeopathic Passiflora incarnata has a potency in the range of 3× to 6×, wherein a dilution of n× dilutes one part of the homeopathic Nux Moschata with nine parts of the diluent and is succussed, and the combination of dilution and succussion are repeated n times. In some preferred embodiments of the composition comprising homeopathically potentised form of Passiflora incarnata and Nux Moschata, the composition includes diluted homeopathic Passiflora incarnata has a potency in the range of 2× to 6×, wherein a dilution of n× dilutes one part of the homeopathic Nux Moschata with nine parts of the diluent and is succussed, and the combination of dilution and succussion are repeated n times. Again, whenever it appears herein, a potency range such as “2× to 6×” refers to each value in the given range up to and including 6×-e.g., “Passiflora incarnata having a potency in the range of 2× to 6× means that Passiflora incarnata may have any one of the potencies 2×, 3×, 4×, 5×, and 6×.

In some exemplary embodiments of the composition comprising homeopathically potentised form of Passiflora incarnata and Nux Moschata, the composition further comprises at least one homeopathically potentised ingredient selected from the group consisting of Aloe Socotrina, Carbo Vegetabilis, Chamomilla, Foeniculum Vulgare, Melissa Officinalis, Mentha Piperita, and Zingiber Officinale.

In some exemplary embodiments of the composition comprising homeopathically potentised form of Passiflora incarnata and Nux Moschata, the composition further comprises at least one ingredient selected from the group consisting of Xylitol, Glycerin, Citric Acid, Potassium Benzoate, Potassium Sorbate, and Potassium Citrate.

In some exemplary embodiments of the composition comprising homeopathically potentised form of Passiflora incarnata and Nux Moschata, the composition further comprises a first homeopathically potentised ingredient, which is at least one member selected from the group consisting of Aloe Socotrina, Carbo Vegetabilis, Chamomilla, Foeniculum Vulgare, Melissa Officinalis, Mentha Piperita, and Zingiber Officinale; and a second ingredient, which is at least one member selected from the group consisting of Xylitol, Glycerin, Citric Acid, Potassium Benzoate, Potassium Sorbate, and Potassium Citrate.

In some preferred embodiments of the composition comprising homeopathically potentised form of Passiflora incarnata and Nux Moschata, the composition further comprises homeopathically potentised Aloe Socotrina, homeopathically potentised Carbo Vegetabilis, homeopathically potentised Chamomilla, homeopathically potentised Foeniculum Vulgare, homeopathically potentised Melissa Officinalis, homeopathically potentised Mentha Piperita, and homeopathically potentised Zingiber Officinale.

In some preferred embodiments of the composition comprising homeopathically potentised form of Passiflora incarnata and Nux Moschata, the composition further comprises Xylitol, Glycerin, Citric Acid, Potassium Benzoate, Potassium Sorbate, and Potassium Citrate.

In some exemplary embodiments of the composition comprising homeopathically potentised form of Passiflora incarnata and Nux Moschata, the composition further comprises a homeopathically potentised Carbo Vegetabilis. In some preferred embodiments, the homeopathically potentised form of the Carbo Vegetabilis is at a potency of 1× or 2× attenuation, triturated in a sugar alcohol such as Xylitol, Mannitol or Sorbitol to between about 10% and about 40% of weight.

In some exemplary embodiments, a composition according to the present invention comprises a homeopathically potentised form of Passiflora incarnata, a homeopathically potentised form of Nux Moschata, and a homeopathically potentised Carbo Vegetabilis. In some preferred embodiments of the composition, the homeopathically potentised form of the Passiflora incarnata is homeopathically diluted to a potency in the range of 3× to 6×, wherein a dilution of n× dilutes one part of the homeopathic Passiflora incarnata with nine parts of a diluent and is succussed, and the combination of dilution and succussion are repeated n times. In some preferred embodiments of the composition, the homeopathically potentised form of the Passiflora incarnata is homeopathically diluted to a potency in the range of 2× to 6×, wherein a dilution of n× dilutes one part of the homeopathic Passiflora incarnata with nine parts of a diluent and is succussed, and the combination of dilution and succussion are repeated n times. Whenever it appears herein, a potency range such as “2× to 6×” refers to each value in the given range up to and including 6×-e.g., “Passiflora incarnata having a potency in the range of 2× to 6× means that Passiflora incarnata may have any one of the potencies 2×, 3×, 4×, 533 , and 6×. In some preferred embodiments of the composition, the homeopathically potentised form of the Carbo Vegetabilis is at a potency of 1× or 2× attenuation, triturated with a sugar alcohol such as Xylitol, Mannitol or Sorbitol to between about 10% and about 40% of weight. In some preferred embodiments of the composition, the homeopathically potentised form of the Nux Moschata is homeopathically diluted to a potency in the range of 3× to 6×, wherein a dilution of n× dilutes one part of the homeopathic Nux Moschata with nine parts of a diluent and is succussed, and the combination of dilution and succussion are repeated n times. In some preferred embodiments of the composition, the homeopathically potentised form of the Nux Moschata is homeopathically diluted to a potency in the range of 2× to 6×, wherein a dilution of n× dilutes one part of the homeopathic Nux Moschata with nine parts of a diluent and is succussed, and the combination of dilution and succussion are repeated n times. In some preferred embodiments of the composition, the homeopathically potentised form of the Nux Moschata has a potency in the range of 3× to 6×, wherein a dilution of n× dilutes one part of the homeopathic the Nux Moschata with nine parts of a diluent and is succussed, and the combination of dilution and succussion are repeated n times; and the homeopathically potentised form of the Carbo Vegetabilis is at a potency of 1× or 2× attenuation, triturated with a sugar alcohol such as Xylitol, Mannitol or Sorbitol to between about 10% and about 40% of weight. In some preferred embodiments of the composition, the homeopathically potentised form of the Nux Moschata has a potency in the range of 2× to 6×, wherein a dilution of n× dilutes one part of the homeopathic the Nux Moschata with nine parts of a diluent and is succussed, and the combination of dilution and succussion are repeated n times; and the homeopathically potentised form of the Carbo Vegetabilis is at a potency of 1× or 2× attenuation, triturated with a sugar alcohol such as Xylitol, Mannitol or Sorbitol to between about 10% and about 40% of weight. In some preferred embodiments of the composition, the homeopathically potentised form of the Passiflora incarnata has a potency in the range of 3× to 6×, wherein a dilution of n× dilutes one part of the homeopathic the Passiflora incarnata with nine parts of a diluent and is succussed, and the combination of dilution and succussion are repeated n times, and the homeopathically potentised form of the Carbo Vegetabilis is at a potency of 1× or 2× attenuation, triturated with a sugar alcohol such as Xylitol, Mannitol or Sorbitol to between about 10% and about 40% of weight. In some preferred embodiments of the composition, the homeopathically potentised form of the Passiflora incarnata has a potency in the range of 2× to 6×, wherein a dilution of n× dilutes one part of the homeopathic the Passiflora incarnata with nine parts of a diluent and is succussed, and the combination of dilution and succussion are repeated n times, and the homeopathically potentised form of the Carbo Vegetabilis is at a potency of 1× or 2× attenuation, triturated with a sugar alcohol such as Xylitol, Mannitol or Sorbitol to between about 10% and about 40% of weight. In some exemplary embodiments, a composition according to the present invention comprises a homeopathically potentised Carbo Vegetabilis, and a homeopathically potentised Passiflora incarnata. In some preferred embodiments of the composition, the homeopathically potentised Carbo Vegetabilis comprises at least one homeopathic dilution. In some preferred embodiments of the composition, each of the homeopathically potentised Carbo Vegetabilis and Passiflora incarnata comprises at least one homeopathic dilution. In some preferred embodiments of the composition, the at least one homeopathic dilution comprises one or more decimal homeopathic dilutions and/or one or more centesimal homeopathic dilutions. In some preferred embodiments of the composition, the composition comprises a diluted homeopathic Nux Moschata having a potency in the range of 3× to 6×, wherein a dilution of n× dilutes one part of the homeopathic Nux Moschata (Nutmeg) with nine parts of the diluent and is succussed, and the combination of dilution and succussion are repeated n times. In some preferred embodiments of the composition, the composition comprises a diluted homeopathic Nux Moschata having a potency in the range of 2× to 6×, wherein a dilution of n× dilutes one part of the homeopathic Nux Moschat (Nutmeg) with nine parts of the diluent and is succussed, and the combination of dilution and succussion are repeated n times. In some preferred embodiments of the composition, the composition comprises a diluted homeopathic Passiflora incarnata having a potency in the range of 3× to 6×, wherein a dilution of n× dilutes one part of the homeopathic Passiflora incarnata with nine parts of the diluent and is succussed, and the combination of dilution and succussion are repeated n times. In some preferred embodiments of the composition, the composition comprises a diluted homeopathic Passiflora incarnata having a potency in the range of 2× to 6×, wherein a dilution of n× dilutes one part of the homeopathic Passiflora incarnata with nine parts of the diluent and is succussed, and the combination of dilution and succussion are repeated n times. In some preferred embodiments of the composition, the composition comprises a diluted homeopathic Carbo Vegetabilis having a potency of 1× or 2×, wherein the Carbo Vegetabilis is triturated with a sugar alcohol such as Xylitol, Mannitol or Sorbitol to between about 10% and about 40% of weight and wherein a dilution of n× dilutes one part of the homeopathic Passiflora incarnata with nine parts of the diluent and is succussed, and the combination of dilution and succussion are repeated n times. In some preferred embodiments of the composition, the composition further comprises at least one homeopathically potentised ingredient selected from the group consisting of Aloe Socotrina, Chamomilla, Foeniculum Vulgare, Melissa Officinalis, Mentha Piperita, and Zingiber Officinale. In some preferred embodiments of the composition, the composition further at least one ingredient selected from the group consisting of Xylitol, Glycerin, Citric Acid, Potassium Benzoate, Potassium Sorbate, and Potassium Citrate.

In some exemplary embodiments, a composition according to the present invention comprises a homeopathically potentised Carbo Vegetabilis, and a homeopathically potentised Nux Moschata. In some preferred embodiments of the composition, the composition includes at least one homeopathic dilution comprising one or more decimal homeopathic dilutions and/or one or more centesimal homeopathic dilutions. In some preferred embodiments of the composition, the composition includes a diluted homeopathic Nux Moschata having a potency in the range of 3× to 6×, wherein a dilution of n× dilutes one part of the homeopathic Nux Moschata with nine parts of the diluent and is succussed, and the combination of dilution and succussion are repeated n times. In some preferred embodiments of the composition, the composition includes a diluted homeopathic Nux Moschata having a potency in the range of 2× to 6×, wherein a dilution of n× dilutes one part of the homeopathic Nux Moschata with nine parts of the diluent and is succussed, and the combination of dilution and succussion are repeated n times. In some preferred embodiments of the composition, the composition includes a diluted homeopathic Carbo Vegetabilis having a potency of 1× or 2×, wherein the Carbo Vegetabilis is triturated with a sugar alcohol such as Xylitol, Mannitol or Sorbitol to between about 10% and about 40% of weight. In some preferred embodiments of the composition, the composition includes a diluted homeopathic Carbo Vegetabilis having a potency of 1× or 2× and a diluted homeopathic Nux Moschata having a potency in the range of 3× to 6×, wherein a dilution of n× dilutes one part of the homeopathic Nux Moschata with nine parts of the diluent and is succussed, and the combination of dilution and succussion are repeated n times. In some preferred embodiments of the composition, the composition includes a diluted homeopathic Carbo Vegetabilis having a potency of 1× or 2× and a diluted homeopathic Nux Moschata having a potency in the range of 2× to 6×, wherein a dilution of n× dilutes one part of the homeopathic Nux Moschata with nine parts of the diluent and is succussed, and the combination of dilution and succussion are repeated n times. In some preferred embodiments of the composition, the composition further comprises at least one homeopathically potentised ingredient selected from the group consisting of Aloe Socotrina, Chamomilla, Foeniculum Vulgare, Melissa Officinalis, Mentha Piperita, and Zingiber Officinale. In some preferred embodiments of the composition, the composition further comprises at least one ingredient selected from the group consisting of Xylitol, Glycerin, Citric Acid, Potassium Benzoate, Potassium Sorbate, and Potassium Citrate. In some preferred embodiments of the composition, the composition further comprises a first homeopathically potentised ingredient, which is at least one member selected from the group consisting of Aloe Socotrina, Chamomilla, Foeniculum Vulgare, Melissa Officinalis, Mentha Piperita, and Zingiber Officinale; and a second ingredient, which is at least one member selected from the group consisting of Xylitol, Glycerin, Citric Acid, Potassium Benzoate, Potassium Sorbate, and Potassium Citrate. In some preferred embodiments of the composition, the composition comprises a homeopathically potentised Carbo Vegetabilis, a homeopathically potentised Nux Moschata, a homeopathically potentised Aloe Socotrina, a homeopathically potentised Carbo Vegetabilis, a homeopathically potentised Chamomilla, a homeopathically potentised Foeniculum Vulgare, a homeopathically potentised Melissa Officinalis, a homeopathically potentised Mentha Piperita; and a homeopathically potentised Zingiber Officinale. In some preferred embodiments of the composition, the composition comprises a homeopathically potentised Carbo Vegetabilis, a homeopathically potentised Nux Moschata, Xylitol, Glycerin, Citric Acid, Potassium Benzoate, Potassium Sorbate; and Potassium Citrate.

Generally, the homeopathically potentised Carbo Vegetabilis in any one of the exemplary and/or preferred compositions according to the present invention can have an iodine number ranging from about 900 mg/g to 2,300 mg/g. In some embodiments, any one of the exemplary and/or preferred compositions according to the present invention can have an iodine number value in a range selected from the group consisting of from about 900 mg/g to about 2300 mg/g, from about 900 mg/g to about 2200 mg/g, from about 900 mg/g to about 2100 mg/g, from about 900 mg/g to about 2000 mg/g, from about 900 mg/g to about 1900 mg/g, from about 900 mg/g to about 1800 mg/g, from about 900 mg/g to about 1700 mg/g, from about 900 mg/g to about 1600 mg/g, from about 900 mg/g to about 1500 mg/g, from about 900 mg/g to about 1400 mg/g, from about 900 mg/g to about 1300 mg/g, from about 900 mg/g to about 1200 mg/g, from about 900 mg/g to about 1100 mg/g, and from about 900 mg/g to about 1000 mg/g.

In some embodiments, any one of the exemplary and/or preferred compositions according to the present invention can have an iodine number value in a range selected from the group consisting of 1000 mg/g to about 2300 mg/g, from about 1000 mg/g to about 2200 mg/g, from about 1000 mg/g to about 2100 mg/g, from about 1000 mg/g to about 2000 mg/g, from about 1000 mg/g to about 1900 mg/g, from about 1000 mg/g to about 1800 mg/g, from about 1000 mg/g to about 1700 mg/g, from about 1000 mg/g to about 1600 mg/g, from about 1000 mg/g to about 1500 mg/g, from about 1000 mg/g to about 1400 mg/g, from about 1000 mg/g to about 1300 mg/g, from about 1000 mg/g to about 1200 mg/g, and from about 1000 mg/g to about 1100 mg/g.

In some embodiments, the homeopathically potentised Carbo Vegetabilis in any one of the exemplary and/or preferred compositions according to the present invention, can have an iodine number value in a range selected from the group consisting of from about 1000 mg/g to about 1400 mg/g, from about 1010 mg/g to about 1400 mg/g, from about 1020 mg/g to about 1400 mg/g, from about 1030 mg/g to about 1400 mg/g, from about 1040 mg/g to about 1400 mg/g, from about 1050 mg/g to about 1400 mg/g, from about 1060 mg/g to about 1400 mg/g, from about 1070 mg/g to about 1400 mg/g, from about 1080 mg/g to about 1400 mg/g, from about 1090 mg/g to about 1400 mg/g, from about 1100 mg/g to about 1400 mg/g, from about 1110 mg/g to about 1400 mg/g, from about 1120 mg/g to about 1400 mg/g, from about 1130 mg/g to about 1400 mg/g, from about 1140 mg/g to about 1400 mg/g, from about 1150 mg/g to about 1400 mg/g, from about 1160 mg/g to about 1400 mg/g, from about 1170 mg/g to about 1400 mg/g, from about 1180 mg/g to about 1400 mg/g, from about 1190 mg/g to about 1400 mg/g, from about 1200 mg/g to about 1400 mg/g, from about 1210 mg/g to about 1400 mg/g, from about 1220 mg/g to about 1400 mg/g, from about 1230 mg/g to about 1400 mg/g, from about 1240 mg/g to about 1400 mg/g, from about 1250 mg/g to about 1400 mg/g, from about 1260 mg/g to about 1400 mg/g, from about 1270 mg/g to about 1400 mg/g, from about 1280 mg/g to about 1400 mg/g, from about 1290 mg/g to about 1400 mg/g, from about 1300 mg/g to about 1400 mg/g, from about 1310 mg/g to about 1400 mg/g, from about 1320 mg/g to about 1400 mg/g, from about 1330 mg/g to about 1400 mg/g, from about 1340 mg/g to about 1400 mg/g, from about 1350 mg/g to about 1400 mg/g, from about 1360 mg/g to about 1400 mg/g, from about 1370 mg/g to about 1400 mg/g, from about 1380 mg/g to about 1400 mg/g, and from about 1390 mg/g to about 1400 mg/g, from about 1410 mg/g to about 2300 mg/g, from about 1420 mg/g to about 2300 mg/g, from about 1430 mg/g to about 2300 mg/g, from about 1440 mg/g to about 2300 mg/g, from about 1450 mg/g to about 2300 mg/g, from about 1460 mg/g to about 2300 mg/g, from about 1470 mg/g to about 2300 mg/g, from about 1480 mg/g to about 2300 mg/g, from about 1490 mg/g to about 2300 mg/g, from about 1500 mg/g to about 2300 mg/g, from about 1510 mg/g to about 2300 mg/g, from about 1520 mg/g to about 2300 mg/g, from about 1530 mg/g to about 2300 mg/g, from about 1540 mg/g to about 2300 mg/g, from about 1550 mg/g to about 2300 mg/g, from about 1560 mg/g to about 2300 mg/g, from about 1570 mg/g to about 2300 mg/g, from about 1580 mg/g to about 2300 mg/g, from about 1590 mg/g to about 2300 mg/g, from about 1600 mg/g to about 2300 mg/g, from about 1610 mg/g to about 2300 mg/g, from about 1620 mg/g to about 2300 mg/g, from about 1630 mg/g to about 2300 mg/g, from about 1640 mg/g to about 2300 mg/g, from about 1650 mg/g to about 2300 mg/g, from about 1660 mg/g to about 2300 mg/g, from about 1670 mg/g to about 2300 mg/g, from about 1680 mg/g to about 2300 mg/g, from about 1690 mg/g to about 2300 mg/g, from about 1700 mg/g to about 2300 mg/g, from about 1710 mg/g to about 2300 mg/g, from about 1720 mg/g to about 2300 mg/g, from about 1730 mg/g to about 2300 mg/g, from about 1740 mg/g to about 2300 mg/g, from about 1750 mg/g to about 2300 mg/g, from about 1760 mg/g to about 2300 mg/g, from about 1770 mg/g to about 2300 mg/g, from about 1780 mg/g to about 2300 mg/g, from about 1790 mg/g to about 2300 mg/g, from about 1800 mg/g to about 2300 mg/g, from about 1810 mg/g to about 2300 mg/g, from about 1820 mg/g to about 2300 mg/g, from about 1830 mg/g to about 2300 mg/g, from about 1840 mg/g to about 2300 mg/g, from about 1850 mg/g to about 2300 mg/g, from about 1860 mg/g to about 2300 mg/g, from about 1870 mg/g to about 2300 mg/g, from about 1880 mg/g to about 2300 mg/g, from about 1890 mg/g to about 2300 mg/g, from about 1900 mg/g to about 2300 mg/g, from about 1910 mg/g to about 2300 mg/g, from about 1920 mg/g to about 2300 mg/g, from about 1930 mg/g to about 2300 mg/g, from about 1940 mg/g to about 2300 mg/g, from about 1950 mg/g to about 2300 mg/g, from about 1960 mg/g to about 2300 mg/g, from about 1970 mg/g to about 2300 mg/g, from about 1980 mg/g to about 2300 mg/g, from about 1990 mg/g to about 2300 mg/g, from about 2000 mg/g to about 2300 mg/g, from about 2010 mg/g to about 2300 mg/g, from about 2020 mg/g to about 2300 mg/g, from about 2030 mg/g to about 2300 mg/g, from about 2040 mg/g to about 2300 mg/g, from about 2050 mg/g to about 2300 mg/g, from about 2060 mg/g to about 2300 mg/g, from about 2070 mg/g to about 2300 mg/g, from about 2080 mg/g to about 2300 mg/g, from about 2090 mg/g to about 2300 mg/g, from about 2100 mg/g to about 2300 mg/g, from about 2110 mg/g to about 2300 mg/g, from about 2120 mg/g to about 2300 mg/g, from about 2130 mg/g to about 2300 mg/g, from about 2140 mg/g to about 2300 mg/g, from about 2150 mg/g to about 2300 mg/g, from about 2160 mg/g to about 2300 mg/g, from about 2170 mg/g to about 2300 mg/g, from about 2180 mg/g to about 2300 mg/g, from about 2190 mg/g to about 2300 mg/g, from about 2200 mg/g to about 2300 mg/g, from about 2210 mg/g to about 2300 mg/g, from about 2220 mg/g to about 2300 mg/g, from about 2230 mg/g to about 2300 mg/g, from about 2240 mg/g to about 2300 mg/g, from about 2250 to about 2300 mg/g, from about 2260 mg/g to about 2300 mg/g, from about 2270 mg/g to about 2300 mg/g, from about 2280 mg/g to about 2300 mg/g, and about 2290 mg/g to about 2300 mg/g.

In some embodiments, the homeopathically potentised Carbo Vegetabilis in any one of the exemplary and/or preferred compositions according to the present invention, can have an iodine number value in a range selected from the group consisting of from at least about 900 mg/g but equal or less than about 2300 mg/g, at least about 910 mg/g but equal or less than about 2300 mg/g, from at least about 920 mg/g but equal or less than about 2300 mg/g, from at least about 930 mg/g but equal or less than about 2300 mg/g, from at least about 940 mg/g but equal or less than about 2300 mg/g, from at least about 950 mg/g but equal or less than about 2300 mg/g, from at least about 960 mg/g but equal or less than about 2300 mg/g, from at least about 970 mg/g but equal or less than about 2300 mg/g, from at least about 980 mg/g but equal or less than about 2300 mg/g, from at least about 990 mg/g but equal or less than about 2300 mg/g, from at least about 1000 mg/g but equal or less than about 1400 mg/g, from at least about 1010 mg/g but equal or less than about 1400 mg/g, from at least about 1020 mg/g but equal or less than about 1400 mg/g, from at least about 1030 mg/g but equal or less than about 1400 mg/g, from at least about 1040 mg/g but equal or less than about 1400 mg/g, from at least about 1050 mg/g but equal or less than about 1400 mg/g, from at least about 1060 mg/g but equal or less than about 1400 mg/g, from at least about 1070 mg/g but equal or less than about 1400 mg/g, from at least about 1080 mg/g but equal or less than about 1400 mg/g, from at least about 1090 mg/g but equal or less than about 1400 mg/g, from at least about 1100 mg/g but equal or less than about 1400 mg/g, from at least about 1110 mg/g but equal or less than about 1400 mg/g, from at least about 1120 mg/g but equal or less than about 1400 mg/g, from at least about 1130 mg/g but equal or less than about 1400 mg/g, from at least about 1140 mg/g but equal or less than about 1400 mg/g, from at least about 1150 mg/g but equal or less than about 1400 mg/g, from at least about 1160 mg/g but equal or less than about 1400 mg/g, from at least about 1170 mg/g but equal or less than about 1400 mg/g, from at least about 1180 mg/g but equal or less than about 1400 mg/g, from at least about 1190 mg/g but equal or less than about 1400 mg/g, from at least about 1200 mg/g but equal or less than about 1400 mg/g, from at least about 1210 mg/g but equal or less than about 1400 mg/g, from at least about 1220 mg/g but equal or less than about 1400 mg/g, from at least about 1230 mg/g but equal or less than about 1400 mg/g, from at least about 1240 mg/g but equal or less than about 1400 mg/g, from at least about 1250 mg/g but equal or less than about 1400 mg/g, from at least about 1260 mg/g but equal or less than about 1400 mg/g, from at least about 1270 mg/g but equal or less than about 1400 mg/g, from at least about 1280 mg/g but equal or less than about 1400 mg/g, from at least about 1290 mg/g but equal or less than about 1400 mg/g, from at least about 1300 mg/g but equal or less than about 1400 mg/g, from at least about 1310 mg/g but equal or less than about 1400 mg/g, from at least about 1320 mg/g but equal or less than about 1400 mg/g, from at least about 1330 mg/g but equal or less than about 1400 mg/g, from at least about 1340 mg/g but equal or less than about 1400 mg/g, from at least about 1350 mg/g but equal or less than about 1400 mg/g, from at least about 1360 mg/g but equal or less than about 1400 mg/g, from at least about 1370 mg/g but equal or less than about 1400 mg/g, from at least about 1380 mg/g but equal or less than about 1400 mg/g, and from at least about 1390 mg/g but equal or less than about 1400 mg/g, from at least about 1410 mg/g but equal or less than about 2300 mg/g, from at least about 1420 mg/g but equal or less than about 2300 mg/g, from at least about 1430 mg/g but equal or less than about 2300 mg/g, from at least about 1440 mg/g but equal or less than about 2300 mg/g, from at least about 1450 mg/g but equal or less than about 2300 mg/g, from at least about 1460 mg/g but equal or less than about 2300 mg/g, from at least about 1470 mg/g but equal or less than about 2300 mg/g, from at least about 1480 mg/g but equal or less than about 2300 mg/g, from at least about 1490 mg/g but equal or less than about 2300 mg/g, from at least about 1500 mg/g but equal or less than about 2300 mg/g, from at least about 1510 mg/g but equal or less than about 2300 mg/g, from at least about 1520 mg/g but equal or less than about 2300 mg/g, from at least about 1530 mg/g but equal or less than about 2300 mg/g, from at least about 1540 mg/g but equal or less than about 2300 mg/g, from at least about 1550 mg/g but equal or less than about 2300 mg/g, from at least about 1560 mg/g but equal or less than about 2300 mg/g, from at least about 1570 mg/g but equal or less than about 2300 mg/g, from at least about 1580 mg/g but equal or less than about 2300 mg/g, from at least about 1590 mg/g but equal or less than about 2300 mg/g, from at least about 1600 mg/g but equal or less than about 2300 mg/g, from at least about 1610 mg/g but equal or less than about 2300 mg/g, from at least about 1620 mg/g but equal or less than about 2300 mg/g, from at least about 1630 mg/g but equal or less than about 2300 mg/g, from at least about 1640 mg/g but equal or less than about 2300 mg/g, from at least about 1650 mg/g but equal or less than about 2300 mg/g, from at least about 1660 mg/g but equal or less than about 2300 mg/g, from at least about 1670 mg/g but equal or less than about 2300 mg/g, from at least about 1680 mg/g but equal or less than about 2300 mg/g, from at least about 1690 mg/g but equal or less than about 2300 mg/g, from at least about 1700 mg/g but equal or less than about 2300 mg/g, from at least about 1710 mg/g but equal or less than about 2300 mg/g, from at least about 1720 mg/g but equal or less than about 2300 mg/g, from at least about 1730 mg/g but equal or less than about 2300 mg/g, from at least about 1740 mg/g but equal or less than about 2300 mg/g, from at least about 1750 mg/g but equal or less than about 2300 mg/g, from at least about 1760 mg/g but equal or less than about 2300 mg/g, from at least about 1770 mg/g but equal or less than about 2300 mg/g, from at least about 1780 mg/g but equal or less than about 2300 mg/g, from at least about 1790 mg/g but equal or less than about 2300 mg/g, from at least about 1800 mg/g but equal or less than about 2300 mg/g, from at least about 1810 mg/g but equal or less than about 2300 mg/g, from at least about 1820 mg/g but equal or less than about 2300 mg/g, from at least about 1830 mg/g but equal or less than about 2300 mg/g, from at least about 1840 mg/g but equal or less than about 2300 mg/g, from at least about 1850 mg/g but equal or less than about 2300 mg/g, from at least about 1860 mg/g but equal or less than about 2300 mg/g, from at least about 1870 mg/g but equal or less than about 2300 mg/g, from at least about 1880 mg/g but equal or less than about 2300 mg/g, from at least about 1890 mg/g but equal or less than about 2300 mg/g, from at least about 1900 mg/g but equal or less than about 2300 mg/g, from at least about 1910 mg/g but equal or less than about 2300 mg/g, from at least about 1920 mg/g but equal or less than about 2300 mg/g, from at least about 1930 mg/g but equal or less than about 2300 mg/g, from at least about 1940 mg/g but equal or less than about 2300 mg/g, from at least about 1950 mg/g but equal or less than about 2300 mg/g, from at least about 1960 mg/g but equal or less than about 2300 mg/g, from at least about 1970 mg/g but equal or less than about 2300 mg/g, from at least about 1980 mg/g but equal or less than about 2300 mg/g, from at least about 1990 mg/g but equal or less than about 2300 mg/g, from at least about 2000 mg/g but equal or less than about 2300 mg/g, from at least about 2010 mg/g but equal or less than about 2300 mg/g, from at least about 2020 mg/g but equal or less than about 2300 mg/g, from at least about 2030 mg/g but equal or less than about 2300 mg/g, from at least about 2040 mg/g but equal or less than about 2300 mg/g, from at least about 2050 mg/g but equal or less than about 2300 mg/g, from at least about 2060 mg/g but equal or less than about 2300 mg/g, from at least about 2070 mg/g but equal or less than about 2300 mg/g, from at least about 2080 mg/g but equal or less than about 2300 mg/g, from at least about 2090 mg/g but equal or less than about 2300 mg/g, from at least about 2100 mg/g but equal or less than about 2300 mg/g, from at least about 2110 mg/g but equal or less than about 2300 mg/g, from at least about 2120 mg/g but equal or less than about 2300 mg/g, from at least about 2130 mg/g but equal or less than about 2300 mg/g, from at least about 2140 mg/g but equal or less than about 2300 mg/g, from at least about 2150 mg/g but equal or less than about 2300 mg/g, from at least about 2160 mg/g but equal or less than about 2300 mg/g, from at least about 2170 mg/g but equal or less than about 2300 mg/g, from at least about 2180 mg/g but equal or less than about 2300 mg/g, from at least about 2190 mg/g but equal or less than about 2300 mg/g, from at least about 2200 mg/g but equal or less than about 2300 mg/g, from at least about 2210 mg/g but equal or less than about 2300 mg/g, from at least about 2220 mg/g but equal or less than about 2300 mg/g, from at least about 2230 mg/g but equal or less than about 2300 mg/g, from at least about 2240 mg/g but equal or less than about 2300 mg/g, from at least about 2250 but equal or less than about 2300 mg/g, from at least about 2260 mg/g but equal or less than about 2300 mg/g, from at least about 2270 mg/g but equal or less than about 2300 mg/g, from at least about 2280 mg/g but equal or less than about 2300 mg/g, and about 2290 mg/g but equal or less than about 2300 mg/g.

In some embodiments, the homeopathically potentised Carbo Vegetabilis in any one of the exemplary and/or preferred compositions according to the present invention, can have an iodine number value in a range selected from the group consisting of at least about 900 mg/g, 910 mg/g, at least about 920 mg/g, at least about 930 mg/g, at least about 940 mg/g, at least about 950 mg/g, at least about 960 mg/g, at least about 970 mg/g, at least about 980 mg/g, at least about 990 mg/g, at least about 1000 mg/g, at least about 1010 mg/g, at least about 1020 mg/g, at least about 1030 mg/g, at least about 1040 mg/g, at least about 1050 mg/g, at least about 1060 mg/g, at least about 1070 mg/g, at least about 1080 mg/g, at least about 1090 mg/g, at least about 1100 mg/g, at least about 1110 mg/g, at least about 1120 mg/g, at least about 1130 mg/g, at least about 1140 mg/g, at least about 1150 mg/g, at least about 1160 mg/g, at least about 1170 mg/g, at least about 1180 mg/g, at least about 1190 mg/g, at least about 1200 mg/g, at least about 1210 mg/g, at least about 1220 mg/g, at least about 1230 mg/g, at least about 1240 mg/g, at least about 1250 mg/g, at least about 1260 mg/g, at least about 1270 mg/g, at least about 1280 mg/g, at least about 1290 mg/g, at least about 1300 mg/g, at least about 1310 mg/g, at least about 1320 mg/g, at least about 1330 mg/g, at least about 1340 mg/g, at least about 1350 mg/g, at least about 1360 mg/g, at least about 1370 mg/g, at least about 1380 mg/g, at least about 1390 mg/g, at least about 1400 mg/g, at least about 1410 mg/g, at least about 1420 mg/g, at least about 1430 mg/g, at least about 1440 mg/g, at least about 1450 mg/g, at least about 1460 mg/g, at least about 1470 mg/g, at least about 1480 mg/g, at least about 1490 mg/g, at least about 1500 mg/g, at least about 1510 mg/g, at least about 1520 mg/g, at least about 1530 mg/g, at least about 1540 mg/g, at least about 1550 mg/g, at least about 1560 mg/g, at least about 1570 mg/g, at least about 1580 mg/g, at least about 1590 mg/g, at least about 1600 mg/g, at least about 1610 mg/g, at least about 1620 mg/g, at least about 1630 mg/g, at least about 1640 mg/g, at least about 1650 mg/g, at least about 1660 mg/g, at least about 1670 mg/g, at least about 1680 mg/g, at least about 1690 mg/g, at least about 1700 mg/g, at least about 1710 mg/g, at least about 1720 mg/g, at least about 1730 mg/g, at least about 1740 mg/g, at least about 1750 mg/g, at least about 1760 mg/g, at least about 1770 mg/g, at least about 1780 mg/g, at least about 1790 mg/g, at least about 1800 mg/g, at least about 1810 mg/g, at least about 1820 mg/g, at least about 1830 mg/g, at least about 1840 mg/g, at least about 1850 mg/g, at least about 1860 mg/g, at least about 1870 mg/g, at least about 1880 mg/g, at least about 1890 mg/g, at least about 1900 mg/g, at least about 1910 mg/g, at least about 1920 mg/g, at least about 1930 mg/g, at least about 1940 mg/g, at least about 1950 mg/g, at least about 1960 mg/g, at least about 1970 mg/g, at least about 1980 mg/g, at least about 1990 mg/g, at least about 2000 mg/g, at least about 2010 mg/g, at least about 2020 mg/g, at least about 2030 mg/g, at least about 2040 mg/g, at least about 2050 mg/g, at least about 2060 mg/g, at least about 2070 mg/g, at least about 2080 mg/g, at least about 2090 mg/g, at least about 2100 mg/g, at least about 2110 mg/g, at least about 2120 mg/g, at least about 2130 mg/g, at least about 2140 mg/g, at least about 2150 mg/g, at least about 2160 mg/g, at least about 2170 mg/g, at least about 2180 mg/g, at least about 2190 mg/g, at least about 2200 mg/g, at least about 2210 mg/g, at least about 2220 mg/g, at least about 2230 mg/g, at least about 2240 mg/g, at least about 2250, at least about 2260 mg/g, at least about 2270 mg/g, at least about 2280 mg/g, at least about 2290 mg/g, and at least about 2300 mg/g.

In some embodiments, the homeopathically potentised Carbo Vegetabilis in any one of the exemplary and/or preferred compositions according to the present invention, can have an iodine number value selected from the group consisting of at least about 900 mg/g, 910 mg/g, about 920 mg/g, about 930 mg/g, about 940 mg/g, about 950 mg/g, about 960 mg/g, about 970 mg/g, about 980 mg/g, about 990 mg/g, about 1000 mg/g, about 1010 mg/g, about 1020 mg/g, about 1030 mg/g, about 1040 mg/g, about 1050 mg/g, about 1060 mg/g, about 1070 mg/g, about 1080 mg/g, about 1090 mg/g, about 1100 mg/g, about 1110 mg/g, about 1120 mg/g, about 1130 mg/g, about 1140 mg/g, about 1150 mg/g, about 1160 mg/g, about 1170 mg/g, about 1180 mg/g, about 1190 mg/g, about 1200 mg/g, about 1210 mg/g, about 1220 mg/g, about 1230 mg/g, about 1240 mg/g, about 1250 mg/g, about 1260 mg/g, about 1270 mg/g, about 1280 mg/g, about 1290 mg/g, about 1300 mg/g, about 1310 mg/g, about 1320 mg/g, about 1330 mg/g, about 1340 mg/g, about 1350 mg/g, about 1360 mg/g, about 1370 mg/g, about 1380 mg/g, about 1390 mg/g, and about 1400 mg/g, about 1410 mg/g, about 1420 mg/g, about 1430 mg/g, about 1440 mg/g, about 1450 mg/g, about 1460 mg/g, about 1470 mg/g, about 1480 mg/g, about 1490 mg/g, about 1500 mg/g, about 1510 mg/g, about 1520 mg/g, about 1530 mg/g, about 1540 mg/g, about 1550 mg/g, about 1560 mg/g, about 1570 mg/g, about 1580 mg/g, about 1590 mg/g, about 1600 mg/g, about 1610 mg/g, about 1620 mg/g, about 1630 mg/g, about 1640 mg/g, about 1650 mg/g, about 1660 mg/g, about 1670 mg/g, about 1680 mg/g, about 1690 mg/g, about 1700 mg/g, about 1710 mg/g, about 1720 mg/g, about 1730 mg/g, about 1740 mg/g, about 1750 mg/g, about 1760 mg/g, about 1770 mg/g, about 1780 mg/g, about 1790 mg/g, about 1800 mg/g, about 1810 mg/g, about 1820 mg/g, about 1830 mg/g, about 1840 mg/g, about 1850 mg/g, about 1860 mg/g, about 1870 mg/g, about 1880 mg/g, about 1890 mg/g, about 1900 mg/g, about 1910 mg/g, about 1920 mg/g, about 1930 mg/g, about 1940 mg/g, about 1950 mg/g, about 1960 mg/g, about 1970 mg/g, about 1980 mg/g, about 1990 mg/g, about 2000 mg/g, about 2010 mg/g, about 2020 mg/g, about 2030 mg/g, about 2040 mg/g, about 2050 mg/g, about 2060 mg/g, about 2070 mg/g, about 2080 mg/g, about 2090 mg/g, about 2100 mg/g, about 2110 mg/g, about 2120 mg/g, about 2130 mg/g, about 2140 mg/g, about 2150 mg/g, about 2160 mg/g, about 2170 mg/g, about 2180 mg/g, about 2190 mg/g, about 2200 mg/g, about 2210 mg/g, about 2220 mg/g, about 2230 mg/g, about 2240 mg/g, about 2250, about 2260 mg/g, about 2270 mg/g, about 2280 mg/g, about 2290 mg/g, and at least about 2300 mg/g.

In some embodiments, the homeopathically potentised Carbo Vegetabilis in any one of the exemplary and/or preferred compositions according to the present invention is sourced from Carbo Vegetabilis powder having a mean particle diameter below about 60 microns. In some embodiments, the homeopathically potentised Carbo Vegetabilis in any one of the exemplary and/or preferred compositions according to the present invention is sourced from Carbo Vegetabilis powder having a mean particle diameter in a range selected from the group consisting of below about 60 microns, below about 65 microns, below about 70 microns, below about 75 microns, below about 80 microns, below about 85 microns, below about 90 microns, below about 95 microns, below about 100 microns, below about 105 microns, below about 110 microns, and below about 115 microns. In some embodiments, the homeopathically potentised Carbo Vegetabilis in any one of the exemplary and/or preferred compositions according to the present invention is sourced from Carbo Vegetabilis powder having a mean particle diameter in a range selected from the group consisting of from 60 microns to about 120 microns, from about 65 microns to about 120 microns, from about 70 microns to about 120 microns, from about 75 microns to about 120 microns, from about 80 microns to about 120 microns, from about 85 microns to about 120 microns, from about 90 microns to about 120 microns, from about 95 microns to about 120 microns, from about 100 microns to about 120 microns, from about 105 microns to about 120 microns, from about 110 microns to about 120 microns, and from about 115 microns to about 120 microns.

In some embodiments, the homeopathically potentised Carbo Vegetabilis in any one of the exemplary and/or preferred compositions according to the present invention is sourced from Carbo Vegetabilis powder having a mean particle diameter below about 60 microns with at least 90% of mass contained in particles below 120 microns diameter. In some embodiments, the homeopathically potentised Carbo Vegetabilis in any one of the exemplary and/or preferred compositions according to the present invention is sourced from Carbo Vegetabilis powder having a mean particle diameter in a range selected from the group consisting of from 60 microns to about 120 microns with at least 90% of mass contained in particles below 120 microns diameter, from about 65 microns to about 120 microns with at least 90% of mass contained in particles below 120 microns diameter, from about 70 microns to about 120 microns with at least 90% of mass contained in particles below 120 microns diameter, from about 75 microns to about 120 microns with at least 90% of mass contained in particles below 120 microns diameter, from about 80 microns to about 120 microns with at least 90% of mass contained in particles below 120 microns diameter, from about 85 microns to about 120 microns with at least 90% of mass contained in particles below 120 microns diameter, from about 90 microns to about 120 microns with at least 90% of mass contained in particles below 120 microns diameter, from about 95 microns to about 120 microns with at least 90% of mass contained in particles below 120 microns diameter, from about 100 microns to about 120 microns with at least 90% of mass contained in particles below 120 microns diameter, from about 105 microns to about 120 microns with at least 90% of mass contained in particles below 120 microns diameter, from about 110 microns to about 120 microns with at least 90% of mass contained in particles below 120 microns diameter, and from about 115 microns to about 120 microns with at least 90% of mass contained in particles below 120 microns diameter.

In some embodiments, any one of the exemplary and/or preferred compositions according to the present invention further comprises diluent, wherein constituent homeopathic ingredients are diluted with the diluent. In some embodiments, any one of the exemplary and/or preferred compositions according to the present invention further comprises a liquid diluent, wherein constituent homeopathic ingredients are diluted with the liquid diluent. In some embodiments, any one of the exemplary and/or preferred compositions according to the present invention further comprises a liquid diluent selected from the group consisting of purified water, strong alcohol, dispensing alcohol, glycerin, and combinations thereof to form diluted homeopathic ingredients. In some embodiments, any one of the exemplary and/or preferred compositions according to the present invention further comprises a liquid diluent comprising water, ethanol, or a combination thereof.

In some embodiments, any one of the exemplary and/or preferred compositions according to the present invention is formulated as an ingestible liquid suspension. “A suspension” as used herein refers to a liquid containing small pieces/particles of one or more of the ingredients of a composition of the present invention, e.g., the small pieces of one or more of the ingredients not completely dissolved in the liquid. In some embodiments, the ingestible liquid suspension of the present invention is preferably shaken (or stirred) vigorously prior to use. Shaking or stirring the ingestible liquid suspension vigorously prior to use ensures that a patient taking the ingestible liquid suspension consistently receives the right amount/dose of the active agent(s) in the ingestible liquid suspension.

The strategy used for selecting the ingredients in the compositions of the present invention employs a combination of remedies which combine several desirable characteristics. In at least one embodiment, the compositions include allopathic amounts of activated carbon (contained in Carbo Vegetabilis 1×) which deliver a weight equivalent therapeutic dose for treatment of gastrointestinal discomfort when allowing for the weight ratio of an infant versus an adult.

In at least one embodiment, the compositions according to the present invention advantageously eliminate or at least minimize any gritty texture, sensation, or feel in the mouth of a subject in need of treatment with these compositions. Compositions that impart gritty texture, sensation, or feel in the mouth of a subject during or following oral administration thereof typically contain suspended particles of activated carbon (typically from Carbo Vegetabilis 1×) that settle on the tongue of the subject. This gritty texture/sensation/feel can be unpleasant and unsettling to an infant to whom the composition is orally administered, and therefore would tend to aggravate rather than sooth colicky infant.

In at least one embodiment, the compositions according to the present invention advantageously eliminate or at least minimize the above-mentioned settling of activated carbon to not only reduce/migate customer rejection of products containing these compositions, but also to demonstrate compliance with the Food and Drug Administration (FDA)'s process validation under 21 C.F.R. §211.110 (i.e. identity, strength, quality, and purity).

In at least one embodiment, the compositions according to the present invention advantageously eliminate or at least minimize the above-mentioned settling of activated carbon to respectively prevent staining of a child's tongue, stool by the activated carbon when orally administered or to prevent staining of the child's clothing if the composition is accidently spilled thereon.

In at least one embodiment, the compositions according to the present invention advantageously eliminate or at least minimize the above-mentioned settling of activated carbon to eliminate or at least minimize dosing errors. Due to its porosity the activated carbon contained in Carbo Vegetabilis 1×, has a density much higher than its apparent density (i.e., its bulk density) and therefore when placed in a non-viscous liquid solution in a container, it will tend to sink to the bottom of the container. This sinking behavior can be avoided if immediately before use, the composition containing Carbo Vegetabilis 1× is shaken vigorously and then applied quickly. However, failure to shake the solution immediately prior to use, can result in the user of the composition making dosing errors, which can result in failure of the user to realize optimal results/effects of the treatment.

In at least one embodiment of the compositions of the present invention, one or both of the ingredients Passiflora incarnata and Nux Moschata are included. Passiflora incarnata at a homeopathic liquid attenuation of 2×, 3×, 4×, 5× or 6× addresses the anxiety aspect as described within the fussy baby theory of colic. Traditionally, passionflower has been used as an herbal medicine to treat nervous anxiety and insomnia. While not previously addressed by any of Colic Calm's ingredients, the fussy baby cause of colic can be alleviated by passionflower. Homeopathic provings and Materia Medica support for Passiflora include Extreme ill-humor; Petulant and irritable; Variable mood; at one time he wishes to do something, but when he is about to accomplish it changes his mind; Scraping eructations, always a short time after eating; Nausea and collection of water in the mouth; Sickness at stomach; Slight burning in the stomach, like a sensation of fasting; and Impaired digestion.

Nux Moschata at a homeopathic liquid attenuation of 2×, 3×, 4×, 5× or 6× addresses gastrointestinal aspects as described within some theories of colic. Traditional medicinal uses of nutmeg include diarrhea, stomach upset, flatulence, and insomnia. Nutmeg has also been shown to have an antimicrobial effect on certain pathogenic bacteria that may be associated with colic.

Analgesic effects have been observed in animal study. Homeopathic provings and Materia Medica support for Nux Moschata include Leaden, dead feeling after or between meals; flatulence and sour eructation, acidity of the stomach; and Pain in the stomach, which comes on an hour or two after taking food.

In some embodiments, any one of the exemplary and/or preferred compositions according to the present invention can contain Carbo Vegetabilis (i.e. Vegetable Charcoal) at 1× or 2× attenuation, the Carbo Vegetabilis being triturated in a sugar alcohol. In some embodiments, the Carbo Vegetabilis is triturated with a sugar alcohol such as Xylitol, Mannitol or Sorbitol. In some embodiments, the Carbo Vegetabilis is triturated with a sugar alcohol such as Xylitol, Mannitol or Sorbitol to between 10% by weight to 40% by weight. In some embodiments, the Carbo Vegetabilis is triturated with a sugar alcohol such as Xylitol, Mannitol or Sorbitol to a proportion selected from the group consisting of about 10% by weight, about 15% by weight, about 20% by weight, about 25% by weight, about 30% by weight, about 35% by weight, and about 40% by weight. In some embodiments, the Carbo Vegetabilis is triturated with a sugar alcohol such as Xylitol, Mannitol or Sorbitol to a proportion having a range selected from the group consisting of from about 10% by weight to about 15% by weight, from about 10% by weight to about 20% by weight, from about 10% by weight to about 25% by weight, from about 10% by weight to about 30% by weight, from about 10% by weight to about 35% by weight, from and about 10% by weight to about 40% by weight.

In some embodiments, any one of the exemplary and/or preferred compositions according to the present invention can contain any combinations of the following ingredients in the listed potencies and dilutions:

Homeopathic Active Ingredients:
Aloe Socotrina (i.e. Aloe) at a homeopathic attenuation of 2×, 3×, 4×, 5× or 6× in water or ethanol, between 0.01% and 10% of weight.
Carbo Vegetabilis (i.e. Vegetable Charcoal) at 1× or 2× attenuation, triturated in a sugar alcohol such as Xylitol, Mannitol or Sorbitol, between 10% and 40% of weight.
Chamomilla (i.e. Chamomile) at a homeopathic attenuation of 2×, 3×, 4×, 5× or 6× in water or ethanol, between 0.01% and 10% of weight.
Foeniculum Vulgare (i.e. Fennel) at a homeopathic attenuation of 2×, 3×, 4×, 5× or 6× in water or ethanol, between 0.01% and 10% of weight.
Melissa Officinalis (i.e. Lemon Balm) at a homeopathic attenuation of 2×, 3×, 4×, 5× or 6× in water or ethanol, between 0.01% and 10% of weight.

1 Mentha Piperita (i.e. Peppermint) at a homeopathic attenuation of 2×, 3×, 4×, 5× or 6× in water or ethanol, between 0.01% and 10% of weight.
Nux Moschata (i.e. Nutmeg) at a homeopathic attenuation of 2×, 3×, 4×, 5× or 6× in water or ethanol, between 0.01% and 10% of weight.
Passiflora Incarnata (i.e. Passion flower) at a homeopathic attenuation of 2×, 3×, 4×, 5× or 6× in water or ethanol, between 0.01% and 10% of weight.
Zingiber Officinale (i.e. Ginger) at a homeopathic attenuation of 2×, 3×, 4×, 5× or 6× in water or ethanol, between 0.01% and 10% of weight.

Inactive Ingredients:
Citric Acid, Glycerin, Benzoic Acid, Potassium Citrate, Purified Water, Sorbic Acid, Xylitol.

### II. DOSAGE AND DOSING REGIMENTS

In certain embodiments, the present invention provides a dosage regimen for administering any one of the compositions according to the present invention to a patient in need of treatment therewith. In at least one embodiment, any one of the exemplary and/or preferred compositions according to the present invention is formulated as a liquid or a liquid suspension. In some preferred embodiments, a dosage regimen comprises administering to a subject in need of treatment therewith a dose ranging from about 0.1 mL to about 10 mL of the liquid or liquid suspension at a frequency ranging from once daily to about 20 times per day. In some preferred embodiments, a dosage regimen comprises administering to a subject in need of treatment therewith a dose selected from the group consisting of about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, about 2 mL, about 2.1 mL, about 2.2 mL, about 2.3 mL, about 2.4 mL, about 2.5 mL, about 2.6 mL, about 2.7 mL, about 2.8 mL, about 2.9 mL, about 3 mL, about 3.1 mL, about 3.2 mL, about 3.3 mL, about 3.4 mL, about 3.5 mL, about 3.6 mL, about 3.7 mL, about 3.8 mL, about 3.9 mL, about 4 mL, about 4.1 mL, about 4.2 mL, about 4.3 mL, about 4.4 mL, about 4.5 mL, about 4.6 mL, about 4.7 mL, about 4.8 mL, about 4.9 mL, about 5 mL, about 5.1 mL, about 5.2 mL, about 5.3 mL, about 5.4 mL, about 5.5 mL, about 5.6 mL, about 5.7 mL, about 5.8 mL, about 5.9 mL, about 6 mL, about 6.1 mL, about 6.2 mL, about 6.3 mL, about 6.4 mL, about 6.5 mL, about 6.6 mL, about 6.7 mL, about 6.8 mL, about 6.9 mL, about 7 mL, about 7.1 mL, about 7.2 mL, about 7.3 mL, about 7.4 mL, about 7.5 mL, about 7.6 mL, about 7.7 mL, about 7.8 mL, about 7.9 mL, about 8 mL, about 8.1 mL, about 8.2 mL, about 8.3 mL, about 8.4 mL, about 8.5 mL, about 8.6 mL, about 8.7 mL, about 8.8 mL, about 8.9 mL, about 9 mL, about 9.1 mL, about 9.2 mL, about 9.3 mL, about 9.4 mL, about 9.5 mL, about 9.6 mL, about 9.7 mL, about 9.8 mL, about 9.9 mL, and about 10 mL at a frequency ranging from once daily to about 20 times per day.

In some preferred embodiments, a dosage regimen comprises administering to a subject in need of treatment therewith a dose having a range selected from the group consisting of from about 0.1 mL to about 10 mL, from about 0.1 mL to about 9.9 mL, from about 0.1 mL to about 9.8 mL, from about 0.1 mL to about 9.7 mL, from about 0.1 mL to about 9.6 mL, from about 0.1 mL to about 9.5 mL, from about 0.1 mL to about 9.4 mL, from about 0.1 mL to about 9.3 mL, from about 0.1 mL to about 9.2 mL, from about 0.1 mL to about 9.1 mL, from about 0.1 mL to about 9 mL, from about 0.1 mL to about 8.9 mL, from about 0.1 mL to about 8.8 mL, from about 0.1 mL to about 8.7 mL, from about 0.1 mL to about 8.6 mL, from about 0.1 mL to about 8.5 mL, from about 0.1 mL to about 8.4 mL, from about 0.1 mL to about 8.3 mL, from about 0.1 mL to about 8.2 mL, from about 0.1 mL to about 8.1 mL, from about 0.1 mL to about 8 mL, from about 0.1 mL to about 7.9 mL, from about 0.1 mL to about 7.8 mL, from about 0.1 mL to about 7.7 mL, from about 0.1 mL to about 7.6 mL, from about 0.1 mL to about 7.5 mL, from about 0.1 mL to about 7.4 mL, from about 0.1 mL to about 7.3 mL, from about 0.1 mL to about 7.2 mL, from about 0.1 mL to about 7.1 mL, from about 0.1 mL to about 7 mL, from about 0.1 mL to about 6.9 mL, from about 0.1 mL to about 6.8 mL, from about 0.1 mL to about 6.7 mL, from about 0.1 mL to about 6.6 mL, from about 0.1 mL to about 6.5 mL, from about 0.1 mL to about 6.4 mL, from about 0.1 mL to about 6.3 mL, from about 0.1 mL to about 6.2 mL, from about 0.1 mL to about 6.1 mL, from about 0.1 mL to about 6 mL, from about 0.1 mL to about 5.9 mL, from about 0.1 mL to about 5.8 mL, from about 0.1 mL to about 5.7 mL, from about 0.1 mL to about 5.6 mL, from about 0.1 mL to about 5.5 mL, from about 0.1 mL to about 5.4 mL, from about 0.1 mL to about 5.3 mL, from about 0.1 mL to about 5.2 mL, from about 0.1 mL to about 5.1 mL, from about 0.1 mL to about 5 mL, from about 0.1 mL to about 4.9 mL, from about 0.1 mL to about 4.8 mL, from about 0.1 mL to about 4.7 mL, from about 0.1 mL to about 4.6 mL, from about 0.1 mL to about 4.5 mL, from about 0.1 mL to about 4.4 mL, from about 0.1 mL to about 4.3 mL, from about 0.1 mL to about 4.2 mL, from about 0.1 mL to about 4.1 mL, from about 0.1 mL to about 4 mL, from about 0.1 mL to about 3.9 mL, from about 0.1 mL to about 3.8 mL, from about 0.1 mL to about 3.7 mL, from about 0.1 mL to about 3.6 mL, from about 0.1 mL to about 3.5 mL, from about 0.1 mL to about 3.4 mL, from about 0.1 mL to about 3.3 mL, from about 0.1 mL to about 3.2 mL, from about 0.1 mL to about 3.1 mL, from about 0.1 mL to about 3 mL, from about 0.1 mL to about 2.9 mL, from about 0.1 mL to about 2.8 mL, from about 0.1 mL to about 2.7 mL, from about 0.1 mL to about 2.6 mL, from about 0.1 mL to about 2.5 mL, from about 0.1 mL to about 2.4 mL, from about 0.1 mL to about 2.3 mL, from about 0.1 mL to about 2.2 mL, from about 0.1 mL to about 2.1 mL, from about 0.1 mL to about 2 mL, from about 0.1 mL to about 1.9 mL, from about 0.1 mL to about 1.8 mL, from about 0.1 mL to about 1.7 mL, from about 0.1 mL to about 1.6 mL, from about 0.1 mL to about 1.5 mL, from about 0.1 mL to about 1.4 mL, from about 0.1 mL to about 1.3 mL, from about 0.1 mL to about 1.2 mL, from about 0.1 mL to about 1.1 mL, from about 0.1 mL to about 1 mL, from about 0.1 mL to about 0.9 mL, from about 0.1 mL to about 0.8 mL, from about 0.1 mL to about 0.7 mL, from about 0.1 mL to about 0.6 mL, from about 0.1 mL to about 0.5 mL, from about 0.1 mL to about 0.4 mL, from about 0.1 mL to about 0.3 mL, from about 0.1 mL to about 0.2 mL, and from about 0.1 mL to about 0.1 mL.

In some embodiments, any one of the dosing regimens can be used for treating or alleviating colic symptoms in an infant. In some embodiments, any one of the dosing regimens can be used for treating, preventing or reducing symptoms associated with infant colic, intestinal gas, bloating, upset stomach, heartburn or indigestion in an infant.

In some embodiments, frequency of dosing is selected from the group consisting of 1 time per day, 2 times per day, 3 times per day, 4 times per day, 5 times per day, 6 times per day, 7 times per day, 8 times per day, 9 times per day, 10 times per day, 11 times per day, 12 times per day, 13 times per day, 14 times per day, 15 times per day, 16 times per day, 17 times per day, 18 times per day, 19 times per day, and 20 times per day.

In some embodiments, frequency of dosing is selected from the group consisting of 1 time to 20 times per day, 2 times to 20 times per day, 3 times to 20 times per day, 4 times to 20 times per day, 5 times to 20 times per day, 6 times to 20 times per day, 7 times to 20 times per day, 8 times to 20 times per day, 9 times to 20 times per day, 10 times to 20 times per day, 11 times to 20 times per day, 12 times to 20 times per day, 13 times to 20 times per day, 14 times to 20 times per day, 15 times to 20 times per day, 16 times to 20 times per day, 17 times to 20 times per day, 18 times to 20 times per day, and 19 times to 20 times per day.

In some embodiments, frequency of dosing is selected from the group consisting of 1 time to 10 times per day, 2 times to 10 times per day, 3 times to 10 times per day, 4 times to 10 times per day, 5 times to 10 times per day, 6 times to 10 times per day, 7 times to 10 times per day, 8 times to 10 times per day, and 9 times to 10 times per day.

In some embodiments, frequency of dosing is selected from the group consisting of 1 times to 6 times per day, 2 times to 6 times per day, 3 times to 6 times per day, 4 times to 6 times per day, and 5 times to 6 times per day.

### III. METHODS OF TREATMENT

In another aspect, the present invention provides for a method of treatment, comprising administering to a subject in need of such treatment a composition according to the present invention. In some embodiments of the method of treatment, the method comprises administering to a subject in need of such treatment any one of the exemplary and/or preferred compositions according to the present invention. In some embodiments of the method of treatment, the method comprises administering to a subject in need of such treatment any one of the exemplary and/or preferred compositions according to the present invention, wherein the treatment comprises treating or alleviating colic symptoms in a subject, e.g., an infant. In some embodiments of the method of treatment, the method comprises administering to a subject in need of such treatment any one of the exemplary and/or preferred compositions according to the present invention, wherein the treatment comprises preventing or reducing symptoms associated with infant colic, intestinal gas, bloating, upset stomach, heartburn or indigestion in a subject, e.g., an infant. In some embodiments of the method of treatment, the method comprises administering to a subject in need of such treatment any one of the exemplary and/or preferred compositions according to the present invention, wherein the treatment comprises preventing or reducing symptoms associated with infant colic, intestinal gas, bloating, upset stomach, heartburn or indigestion in a pediatric population. In some embodiments of the method of treatment, the method comprises administering to a subject in need of such treatment any one of the exemplary and/or preferred compositions according to the present invention, wherein the composition is present in an amount effective for treating, preventing, or reducing colic or symptoms associated with colic, intestinal gas, bloating, upset stomach, heartburn or indigestion in a subject. In some embodiments of the method of treatment, the method comprises administering to a subject in need of such treatment any one of the exemplary and/or preferred compositions according to the present invention, wherein the composition is present in an amount effective for treating subject who is an infant.

The invention disclosed herein is illustrated in the following non-limiting examples.

### IV. EXAMPLES

### Example 1

### Exemplary Composition 1 of Ingredients

| | | | | |
|---|---|---|---|---|
| | | | Amount of | Amount of |
| | | | Diluted | Undiluted |
| Homeopathic | Homeopathic | Purpose/Homeopathic | Ingredient (% | Ingredient (% |
| Component | Attenuation | Indications | w/w)* | w/w)** |

Active Ingredients:

| | | | | |
|---|---|---|---|---|
| Aloe Socotrina | 3X | Gas Relief, Bloating | 8.3% | 0.0083% |
| Carbo Vegetabilis | 1X | Gas Relief, Upset Stomach | 28% | 2.8% |
| Chamomilla | 3X | Irritability, Hiccups | 8.3% | 0.0083% |
| Foeniculum | 3X | Gas Relief | 8.3% | 0.0083% |
| Vulgare | | | | |
| Melissa Officinalis | 3X | Irritability, Restlessness | 8.3% | 0.0083% |
| Mentha Piperita | 3X | Bloating, Colic | 8.3% | 0.0083% |
| Nux Moschata | 3X | Upset Stomach, Irritability | 8.3% | 0.0083% |
| Passiflora | 3X | Gas Relief, Upset Stomach | 8.3% | 0.0083% |
| Incarnata | | | | |
| Zingiber Officinale | 3X | Digestive Aid | 8.3% | 0.0083% |

Inactive Ingredients:

| | | | | |
|---|---|---|---|---|
| Purified Water | n/a | Carrier, Suspension, | 0%*** | 66.33% |
| | | Attenuation, Diluent | | |
| Xylitol | n/a | Attenuation, Diluent, Taste | 0%*** | 25.2% |
| Glycerin | n/a | Preservative, Taste | 4.85% | 4.85% |
| Citric Acid | n/a | Preservative, Taste | 0.5% | 0.5% |
| Potassium | n/a | Preservative | 0.1% | 0.1% |
| Benzoate | | | | |
| Potassium Sorbate | n/a | Preservative | 0.1% | 0.1% |
| Potassium Citrate | n/a | Preservative | 0.05% | 0.05% |

*The amount of ingredient refers to the total weight percent of the homeopathic active ingredient inclusive of the diluent (either diluted in water or ethanol/water combination or diluted through trituration with an alcohol sugar such as xylitol).
**The amount of ingredient refers to the total weight percent of the homeopathic active ingredient only (i.e. not inclusive of diluent), in the composition as a whole.
***The amount of diluent is additional to that required after the homeopathic active ingredient is diluted to the designated homeopathic dilution.

### Example 2

### Exemplary Composition 2 of Ingredients

| | | | | |
|---|---|---|---|---|
| | | | Amount of | Amount of |
| | | | Diluted | Undiluted |
| Homeopathic | Homeopathic | Purpose/Homeopathic | Ingredient (% | Ingredient (% |
| Component | Attenuation | Indications | w/w)* | w/w)** |

Active Ingredients:

| | | | | |
|---|---|---|---|---|
| Aloe Socotrina | 5X | Gas Relief, Bloating | 5.00% | 0.00005% |
| Carbo Vegetabilis | 1X | Gas Relief, Upset Stomach | 15% | 1.50% |
| Chamomilla | 5X | Irritability, Hiccups | 5.00% | 0.00005% |
| Foeniculum | 5X | Gas Relief | 5.00% | 0.00005% |
| Vulgare | | | | |
| Melissa Officinalis | 5X | Irritability, Restlessness | 5.00% | 0.00005% |
| Mentha Piperita | 5X | Bloating, Colic | 5.00% | 0.00005% |
| Nux Moschata | 5X | Upset Stomach, Irritability | 5.00% | 0.00005% |
| Passiflora | 5X | Gas Relief, Upset Stomach | 5.00% | 0.00005% |
| Incarnata | | | | |
| Zingiber Officinale | 5X | Digestive Aid | 5.00% | 0.00005% |

Inactive Ingredients:

| | | | | |
|---|---|---|---|---|
| Purified Water | n/a | Carrier, Suspension, | 27.7%*** | 67.70% |
| | | Attenuation, Diluent | | |
| Xylitol | n/a | Attenuation, Diluent, Taste | 11.7%*** | 25.20% |
| Glycerin | n/a | Preservative, Taste | 4.85% | 4.85% |
| Citric Acid | n/a | Preservative, Taste | 0.50% | 0.50% |
| Potassium | n/a | Preservative | 0.10% | 0.10% |
| Benzoate | | | | |
| Potassium Sorbate | n/a | Preservative | 0.10% | 0.10% |
| Potassium Citrate | n/a | Preservative | 0.05% | 0.05% |

*The amount of ingredient refers to the total weight percent of the homeopathic active ingredient inclusive of the diluent (either diluted in water or ethanol/water combination or diluted through trituration with an alcohol sugar such as xylitol).
**The amount of ingredient refers to the total weight percent of the homeopathic active ingredient only (i.e. not inclusive of diluent), in the composition as a whole.
***The amount of diluent is additional to that required after the homeopathic active ingredient is diluted to the designated homeopathic dilution.

### Example 3

### Trituration of Carbo Vegetibilis

The HPUS states that lactose monohydrate is the preferred vehicle for the preparation of triturations. However, the present invention uses xylitol or any other other alcohol sugar instead of lactose to triturate Carbo Vegitabilis 1× for the following reasons:
Lactose can cause excess intestinal gas in children, particularly with the presence of gas producing bacteria, which can make the condition of colic worse (see causes). Sugar alcohols such as Xylitol are not associated with increased gassiness.
Xylitol exhibits anti-microbial properties. Studies have shown reductions in tooth decay as well as a reduction in the number of sinus and ear infections with the use of Xylitol.
Rapid changes in blood sugar can cause mood altering effects. Unlike lactose, xylitol and other alcohol sugars do not cause a spike in blood sugar. In effect the product is sugar free, a highly desirable feature with today's consumers.

Some babies may have a condition called Transient Lactase Deficiency (TLD) which inhibits their ability to digest lactose and may actually be a cause of colic.

### Example 4

### Use of a Specific Type of Activated Charcoal for Carbo Vegetabilis 1×, in Place of Generic Vegetable Charcoal

Generic USP grade (latin: Carbo Vegetabilis) vegetable charcoal contained in Carbo Vegetabilis can be made from many types of vegetable matter including wood, nutshells, peat, lignite, coal and petroleum pitch. The type of charcoal carbon is not specified in the HPUS and the HPUS makes no distinction between its various forms simply describing Carbo Vegetabilis as “the residue from the destructive distillation (carbonization) of various vegetable matter, treated to increase its adsorptive power. However, the inventors are aware that the medicinal properties of vegetable charcoal in treating colic stem from its micro-porosity. The adsorptive capacity of vegetable charcoal is generally measured through its iodine number which typically ranges from 500-1000 mg/g depending on the quality and grade of charcoal. However, the present invention uses a special type of super-activated carbon with an Iodine number exceeding 1,400 mg/g. Produced from coconut shells or hard wood, the super-activated carbon used in the present invention undergoes multiple acid baths to improve its porosity. In this application the use of super-activated charcoal with an Iodine number above 1,400 mg/g increases the potency and consequently enables a reduced quantity of Carbo Vegetabilis 1× to be used for the same medicinal effect. This in-turn reduces the disadvantages previously discussed, namely; staining of clothing and tongue, and darkening of stools. It also reduces the amount of Xylitol required to triturate the Carbo Vegetabilis. Xylitol is currently the most costly ingredient in the formula.

### Example 5

### Use of a Specific Particle Size of Activated Charcoal for Carbo Vegetabilis 1×

The HPUS does not prescribe the type or particles size of activated charcoal to be used in the preparation of Carbo Vegetabilis 1×. However, as described elsewhere in this disclosure, conventional activated charcoal can impart undesirable characteristics to a composition, including gritty texture and feel in the mouth when orally administered. Through experimentation, the inventors have surprisingly found that use of activated carbon powder having a mean particle diameter of between 15 microns and 60 microns optimizes efficacy, minimizes staining and improves suspension during manufacturing and therapeutic use with infants experiencing gastrointestinal distress. Carbo Vegetabilis meeting these requirements can be obtained from hardwood, fruits and nuts.

Maximum Size of Particles

When the following specifications are met; activated carbon particle size with a mean particle diameter (MPD) below 60 microns with at least 90% of mass contained in particles (% <d90) below 120 microns diameter, the inventors observed the following advantages:
Improved suspension in the mixture as described in Colic Calm formulation. Upon agitation of the formula, the carbon particles remain in liquid suspension for a much longer period of time ensuring:
   Improved consistency during manufacturing in order to comply with process validation requirements outlined in FDA 21 CFR 211.110 (identity, strength, quality, and purity).
   More consistent dosage delivery when shaken and used by consumers.
Acceleration of efficacy—the speed by which activated carbon exhibits its medicinal properties is related to the particle size. Smaller particles will present a greater external surface in proportion to their weight thus enabling more rapid adsorption of certain organic gasses contained in the GI tract.

Minimum Size of Particles

When the following specifications are met; activated carbon particle size with a mean particle diameter (MPD) above 15 microns with less than 10% of mass contained in particles (% <d10) below 4 microns diameter, the inventors observed the following advantages:

Less staining of clothing—larger charcoal particles exhibit a diminished propensity to adhere to the fibers of clothing.

Less staining of tongue—larger charcoal particles exhibit a diminished propensity to adhere to the tongue.

Less stool darkening—larger charcoal particles exhibit a smaller surface area when present in stool.

Duration of action—if particles are too small the medicinal properties of activated charcoal become spent before the particles have had time to travel to the upper or lower intestines where gas pain is typically manifested since larger particles exhibit medicinal properties more slowly but over a long period of time.

It will be appreciated by those skilled in the art that changes could be made to the exemplary embodiments shown and described above without departing from the broad inventive concept thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and features of the disclosed embodiments may be combined. Unless specifically set forth herein, the terms “a”, “an” and “the” are not limited to one element but instead should be read as meaning “at least one”.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the method does not rely on the particular order of steps set forth herein, the particular order of the steps should not be construed as limitation on the claims. The claims directed to the method of the present invention should not be limited to the performance of their steps in the order written, and one skilled in the art can readily appreciate that the steps may be varied and still remain within the spirit and scope of the present invention.

I/we claim:

1-42. (canceled)

43. A homeopathic composition comprising homeopathically potentised Carbo Vegetabilis formulated in a liquid suspension.

44. The homeopathic composition of claim 43, wherein the homeopathically potentised form of the Carbo Vegetabilis is at a potency of 1× or 2×.

45. The homeopathic composition of claim 43, wherein the Carbo Vegetabilis is triturated with a sugar alcohol selected from the group consisting of Xylitol, Mannitol and Sorbitol or a combination thereof.

46. The homeopathic composition of claim 43, wherein the Carbo Vegetabilis is produced from activated carbon having an iodine number of at least 900 mg/g.

47. The homeopathic composition of claim 43, wherein the Carbo Vegetabilis comprises activated carbon particles having a mean particle size of about 15 microns to about 60 microns with at least 90% of mass contained in particles (% <d90) below 120 microns diameter.

48. The homeopathic composition of claim 43, further comprising homeopathically potentised Passiflora Incarnata in the range of 2× to 6× and/or homeopathically potentised Nux Moschata in the range of 2× to 6×.

49. The homeopathic composition of claim 43, further comprising three or more homeopathically potentised ingredients with potency in the range of 2× to 6× selected from the following list: Passiflora Incarnata, Nux Moschata, Aloe Socotrina, Chamomilla, Foeniculum Vulgare, Melissa Officinalis, Mentha Piperita, and Zingiber Officinale.

50. The homeopathic composition of claim 43, further comprising at least one of the following ingredients: Xylitol, Glycerin, Citric Acid, Potassium Benzoate, Potassium Sorbate, and Potassium Citrate.

51. The homeopathic composition of claim 49, wherein the Passiflora incarnata has a 3× potency, the Nux Moschata has a 3× potency, the Aloe Socotrina has a 3× potency, the Carbo Vegetabilis has a 1× potency, the Chamomilla has a 3× potency, the Foeniculum Vulgare has a 3× potency, the Melissa Officinalis has a 3× potency, the Mentha Piperita has a 3× potency, and the Zingiber Officinale has a 3× potency.

52. The homeopathic composition of claim 49, wherein the Passiflora incarnata has a 5× potency, the Nux Moschata has a 5× potency, the Aloe Socotrina has a 5× potency, the Carbo Vegetabilis has a 1× potency, the Chamomilla has a 5× potency, the Foeniculum Vulgare has a 5× potency, the Melissa Officinalis has a 5× potency, the Mentha Piperita has a 5× potency, and the Zingiber Officinale has a 5× potency.

53. A method of treatment, comprising administering to a subject in need of such treatment a composition according to claim 43, wherein the homeopathic composition is present in an amount effective for treating, preventing, or reducing symptoms associated with infant colic, intestinal gas, bloating, upset stomach, heartburn or indigestion in a pediatric population.

54. A composition comprising vegetable charcoal formulated in a liquid suspension at a concentration between 0.1% and 10%.

55. The composition of claim 54, where the vegetable charcoal comprises activated carbon particles having a mean particle size of about 15 microns to about 60 microns with at least 90% of mass contained in particles (% <d90) below 120 microns diameter.

56. The composition of claim 54, further comprising a sugar alcohol selected from the group consisting of xylitol, mannitol and sorbitol, and further comprising two or more herbal extracts selected from the following list: passionflower, nutmeg, aloe, chamomile, fennel, lemon balm, peppermint, ginger.

57. A method of treatment, comprising administering to a subject in need of such treatment a composition according to claim 54, wherein the composition is present in an amount effective for treating, preventing, or reducing symptoms associated with infant colic, intestinal gas, bloating, upset stomach, heartburn or indigestion in a pediatric population.
